SEQUENCE LISTING (26 sequences)

SEQ ID NO: 1 - protein, 12 aa, Artificial Sequence
  NGN family  domain in an artificial polypeptide
XXXKXXDXXXNX

SEQ ID NO: 2 - protein, 12 aa, Artificial Sequence
  ATO family - domain in an artificial polypeptide
XXXAXXAXXXRX

SEQ ID NO: 3 - protein, 12 aa, Artificial Sequence
  domain in an artificial polypeptide
RRMKANDRERNR

SEQ ID NO: 4 - protein, 12 aa, Artificial Sequence
  domain in an artificial polypeptide
RRLAANARERRR

SEQ ID NO: 5 - protein, 10 aa, Artificial Sequence
  consensus sequence
XKIETLRXAX

SEQ ID NO: 6 - protein, 10 aa, Artificial Sequence
  Neurogenin
TKIETLRFAX

SEQ ID NO: 7 - protein, 10 aa, Artificial Sequence
  NeuroD
SKIETLRLAK

SEQ ID NO: 8 - protein, 12 aa, Artificial Sequence
  domain in an artificial polypeptide
RRVAANARERRR

SEQ ID NO: 9 - protein, 12 aa, Artificial Sequence
  domain in an artificial polypeptide
RRLKANDRERNR

SEQ ID NO: 10 - protein, 57 aa, Drosophila melanogaster
  MISC_FEATURE: TAP
RRMKANDRERNRMHNLNDALEKLRVTLPSLPEETKLTKIEILRFAHNYIFALEQVLE

SEQ ID NO: 11 - protein, 57 aa, Xenopus laevis
  MISC_FEATURE: NGN1
RRVKANDRERNRMHNLNAALDALRSVLPSFPDDTKLTKIETLRFAYNYIWALAETLR

SEQ ID NO: 12 - protein, 57 aa, Drosophila melanogaster
  MISC_FEATURE: ATO
RRLAANARERRRMQNLNQAFDRLRQYLPCLGNDRQLSKHETLQMAQTYISALGDLLR

SEQ ID NO: 13 - protein, 57 aa, Mus musculus
  MISC_FEATURE: MATH1
RRLAANARERRRMHGLNHAFDQLRNVIPSFNNDKKLSKYETLQMAQIYINALSELLQ

SEQ ID NO: 14 - protein, 45 aa, Drosophila melanogaster
  MISC_FEATURE: ATO
MQNLNQAFDRLRQYLPCLGNDRQLSKHETLQMAQTYISALGDLLR

SEQ ID NO: 15 - protein, 45 aa, Drosophila melanogaster
  misc_feature: AMOS
MNSLNDAFDKLRDVVPSLGHDRRLSKYETLQMAQAYIGDLVTLLS

SEQ ID NO: 16 - protein, 45 aa, Drosophila melanogaster
  misc_feature: CATO
MNGLNAAFERLREVVPAPSIDQKLSKFETLQMAQSYILALCDLLN

SEQ ID NO: 17 - protein, 45 aa, Mus musculus
  misc_feature: MATH1
MHGLNHAFDQLRNVIPSFNNDKKLSKYETLQMAQIYINALSELLQ

SEQ ID NO: 18 - protein, 45 aa, Mus musculus
  misc_feature: MATH5
MQGLNTAFDRLRRVVPQWGQDKKLSKYETLQMALSYIIALTRILA

SEQ ID NO: 19 - protein, 45 aa, Drosophila melanogaster
  misc_feature: Biparous
MHNLNDALEKLRVTLPSLPEETKLTKIEILRFAHNYIFALEQVLE

SEQ ID NO: 20 - protein, 45 aa, Xenopus laevis
  misc_feature: XNGN1
MHNLNSALDSLREVLPSLPEDAKLTKIETLRFAYNYIWALSETLR

SEQ ID NO: 21 - protein, 45 aa, Mus musculus
  misc_feature: MNGN1
MHNLNAALDALRSVLPSFPDDTKLTKIETLRFAYNYIWALAETLR

SEQ ID NO: 22 - protein, 45 aa, Mus musculus
  misc_feature: MNGN2
MHNLNAALDALREVLPTFPEDAKLTKIETLRFAHNYIWALTETLR

SEQ ID NO: 23 - protein, 45 aa, Mus musculus
  misc_feature: MNGN3
MHNLNSALDALRGVLPTFPDDAKLTKIETLRFAHNYIWALTQTLR

SEQ ID NO: 24 - protein, 45 aa, Danio rerio
  misc_feature: ZNGN2
MHNLNDALDALRSVLPAFPDDTKLTKIETLRFAHNYIWALSETIR

SEQ ID NO: 25 - protein, 10 aa, Artificial Sequence
  domain in an artificial polypeptide
SKHETLQMAQ

SEQ ID NO: 26 - protein, 10 aa, Artificial Sequence
  domain in an artificial polypeptide
TKIETLRFAY

(19) United States
(12) Patent Application Publication
(10) Pub. No.: US 2006/0019386 A1
(43) Pub. Date: Jan. 26, 2006

(54) Protein domain related to deafness, osteoarthritis and abnormal cell proliferation

(75) Inventors: Hassan Bassem, Linkebeek (BE); Xiao-Jiang Quan, Auderghem (BE); Wouter Bossuyt, Kuurne (BE)

Correspondence Address: TRASK BRITT, P.O. BOX 2550, SALT LAKE CITY, UT, 84110, US

(73) Assignees: VIB vzw, Zwijnaarde (BE); K.U. LEUVEN RESEARCH & DEVELOPMENT, Leuven (BE)

(21) Appl. No.: 11/186,545

(22) Filed: Jul. 21, 2005

Related U.S. Application Data
(63) Continuation of application No. PCT/EP04/50033, filed on Jan. 21, 2004.

(30) Foreign Application Priority Data
Jan. 21, 2003 (EP) 03100110.0
May 27, 2003 (EP) 03101542.3

(51) Int. Cl. C12N 5/08; A61K 38/10

(52) U.S. Cl. 435/368; 514/14; 530/328

(57) ABSTRACT

The present invention relates to genetic diagnosis and therapy of diseases of the nervous system (NS). More particularly, it relates to methods to induce neural precursor cells (NPCs) and to the identification of a domain that determines the functionality of polypeptides belonging to the atonal family and its use in therapy for the treatment of deafness, partial hearing loss and vestibular defects due to damage of loss of inner ear hair cells. Alternatively, the domain may be used in the treatment of cancer.

### CROSS-REFERENCE TO RELATED APPLICATIONS

This application is a continuation of PCT International Patent Application No. PCT/EP2004/050033, filed on Jan. 21, 2004, designating the United States of America, and published, in English, as PCT International Publication No. WO 2004/065538 A2 on Aug. 5, 2004, which application claims priority to European Patent Application Serial No. 03101542.3 filed on May 27, 2003, which application in turn claims priority to European Patent Application Serial No. 03100110.0 filed on Jan. 21, 2003, the contents of the entirety of each of which are incorporated by this reference.

### TECHNICAL FIELD

Embodiments of the invention generally relate to biotechnology and to genetic diagnosis and therapy of diseases of the nervous system (NS). More particularly, various embodiments relate to methods to induce neural precursor cells (NPCs) and to the identification of a domain that determines the functionality of polypeptides belonging to the atonal-related proteins, and its use in therapy for the treatment of deafness, partial hearing loss and vestibular defects due to damage of loss of inner ear hair cells. Alternatively, the domain may be used in the treatment of cancer.

### BACKGROUND

Damage to hair cells in the ear is a common cause of deafness and vestibular dysfunction, which are themselves prevalent diseases. In the United States, over 28 million people have impaired hearing; vestibular disorders affect about one quarter of the general population and about half of the elderly. The delicate hair cells are vulnerable to disease, aging and environmental trauma, such as the use of antibiotics, or persistent loud noise. In mammals, these cells cannot regenerate once they have been destroyed. WO0073764 discloses how these problems can be addressed, by the use of an atonal associated sequence that plays a crucial role in the development.

The development of multicellular organisms, including the development of specialized organs, involves a complex interplay between factors, which regulate genes transcription and mediate cell-cell interaction, many of which define genetic pathways that are evolutionarily conserved. Although it is conceptually clear that different mechanisms caused by differential interactions among highly conserved proteins result in dramatically different outcomes, little is known about the genetic and molecular basses of these differences. An interesting example is the one used in both vertebrate and invertebrate embryos to select neural precursor cells (NPCs) at early steps in the development of cell lineages.

The initiation event in neural lineage development is the selection and the specification of NPCs. Working in the peripheral nervous system (PNS) of various model systems, such as Drosophila, Xenopus and mouse, has proven that expression of proneural genes in the neuroectoderm is believed to confer the ability to give rise to neural precursors. Proneural polypeptides are a subset of transcription factors of the Basic Helix-Loop-Helix (bHLH) super-family. The proneural polypeptides promote NPC formation by forming heterodimers with a ubiquitously expressed bHLH protein (called Daughterless in Drosophila, and E12/E47 in vertebrates) and activating transcription of target genes via binding to a DNA motif, the E-box, with the basic domain. The function of bHLH proteins is thought to reside mostly within the bHLH domain, a stretch of 57 amino acids residues.

Expression of proneural genes also regulates a lateral inhibition process mediated by Notch signaling pathway via local cell-cell interaction (reviewed in Artavanis-Tsakonas et al., 1995). Activation of Notch receptor ligands, such as Delta, is under the transcriptional control of proneural genes and leads to an intra-membrane cleavage, which release the Notch intracellular domain. The translocation of Notch intracellular domain into the nucleus represses proneural genes by activating the expression of the Enhancer of split E(spl) complex (Bailey and Posakony, 1995; Jennings et al., 1995; Lecourtois and Schweisguth, 1995). The genes required for these steps are highly conserved structurally and functionally between Drosophila and vertebrates.

Two families of proneural bHLH proteins have been found and are conserved across species: the Achaete-Scute proteins (AS) and the Atonal-related proteins (ARPs) (Bertrand et al., 2002; Hassan and Bellen, 2000). The ARPs consist of several subgroups, NeuroD, Neurogenin (NGN) and Atonal (ATO) group (FIG. 1, Panel A). NeuroD proteins are not required for NPC selection in vertebrates and no members of this group exist in flies. In contrast, NGN and ATO group proteins act as proneural polypeptides at the earliest steps of NPC selection (Fode et al., 1998; Goulding et al., 2000; Huang et al., 2000; Jarman et al., 1993; Ma et al., 1996). Gene substitution and mis-expression studies between ATO group proteins, within and across species, suggest that there is a very high degree of functional similarity, and sometimes even functional identity (Ben-Arie et al., 2000; Goulding et al., 2000; Wang et al., 2002). Although this has not been directly tested by gene replacement, expression and mutant analyses suggest that it may be true for the NGN group as well (Begbie et al., 2002; Ma et al., 1999). Both flies and vertebrates have NS-expressed genes belonging to the NGN and ATO groups. These two groups of polypeptides show 47% identity and 58% similarity in bHLH domain (FIG. 1). Interestingly, TAP, the fly NGN group protein is not expressed during NPC selection in the fly PNS and does not appear to have proneural activity (Bush et al., 1996; Gautier et al., 1997). Conversely, ATO proteins are generally not expressed during early NPC selection in vertebrate neural plate (Ben-Arie et al., 2000; Brown et al., 1998; Helms et al., 2001; Kanekar et al., 1997; Kim et al., 1997). Therefore, it is possible to ask whether this reversal in the requirement of proneural genes in NPC selection represents a divergence in the mechanisms by which NPCs are specified, or a simple inert change in expression patterns.

### SUMMARY OF THE INVENTION

To examine this question a comparative study of the proneural capacities of ATO and NGN group proteins was initiated using Drosophila and Xenopus as model organisms. Surprisingly we found that ATO group proteins, potent inducers of NPCs in the fly, are extremely weak inducers of NPCs in Xenopus. In contrast, NGN proteins, potent inducers of NPCs in vertebrates, are, surprisingly, extremely weak inducers in flies. In various embodiments of this invention, we identified the specific residues and structural motifs responsible for proneural activity in each protein and showed that they mediate the specificity of the genetic interactions with the appropriate Zn finger proteins. The difference between the two group polypeptides is not due to the fact that these proteins recognize and interact with only their specific Daughterless family proteins or Notch signaling pathway. A zinc finger transcription factor Senseless (SENS) is essential for the proneural activity of ATO polypeptides in Drosophila, whereas it is not responsive to NGN polypeptides. Conversely, the zinc finger protein MyT1 is essential for the proneural activity of NGN polypeptides in vertebrates, whereas it is not responsive to ATO polypeptides. Even more surprisingly, we were able to prove that the proneural specificity of these two groups of polypeptides resides in three non-DNA contact residues within the basic domain. Exchanging only these three residues can exchange the proneural specificity between these two groups of polypeptides. In summary, we identify both extrinsic and intrinsic factors responsible for specificity of NPC selection.

In an embodiment of the invention is a biological active artificial polypeptide comprising a domain selected from the group consisting of SEQ ID NO:1, SEQ ID NO:2 and SEQ ID NO:5. In an alternate embodiment is an artificial polypeptide according to the invention, whereby the domain consists of SEQ ID NO:3. An alternate embodiment is an artificial polypeptide according to the invention, whereby the domain consists of SEQ ID NO:4. Still another embodiment is an artificial polypeptide according to the invention, whereby the domain consists of SEQ ID NO:6. Still another embodiment is an artificial polypeptide according to the invention, whereby the domain consists of SEQ ID NO:7.

Other embodiments of the invention are the use of an artificial polypeptide according to the invention to modulate neural precursor cell selection and/or to program stem cells. Indeed, it was shown that the specified domain of the invention is determining the neural precursor selection. Overexpression of a polypeptide, comprising an active domain will stimulate NPC formation, whereas overexpression of a polypeptide comprising an inactive domain will have an inhibitory action.

Still another embodiment of the invention is the use of an antibody against a domain selected from the group consisting of SEQ ID NO:1, SEQ ID NO:2 and SEQ ID NO:5 to inhibit neural precursor cell selection. In various embodiments, the antibody is directed against a domain consisting of SEQ ID NO:3, SEQ ID NO:4, SEQ ID NO:6 or SEQ ID NO:7. Indeed, in various embodiments, as the biological activity of the polypeptide is determined by the domains, antibody binding will inhibit the normal interactions of the domain and block the normal biological function. The antibodies can be polyclonal or monoclonal antibodies. Methods to isolate antibodies directed to a specified domain are known to the person skilled in the art.

Another embodiment of the invention is the use of a polypeptide comprising a domain selected from the group consisting of SEQ ID NO:1, SEQ ID NO:2, SEQ ID NO:3, SEQ ID NO:4, SEQ ID NO:5, SEQ ID NO:6 and SEQ ID NO:7 to specify the neuronal lineage identity of stem cells.

Still another embodiment of the invention is the use of a polypeptide comprising a domain selected from the group consisting of SEQ ID NO:1, SEQ ID NO:2, SEQ ID NO:3, SEQ ID NO:4, SEQ ID NO:5, SEQ ID NO:6 and SEQ ID NO:7 to select inhibitors against the biological activity of the domain. In an embodiment, the polypeptide is an artificial polypeptide. As the biological activity of the polypeptide can be measured either in Xenopus cells, or in Drosophila, it is possible to screen for compounds that block the biological activity. Such compounds are, as a non-limiting example, polypeptides that are interacting with the domain, or peptido-mimetics of an inactive domain.

Another embodiment of the invention is the use of a polypeptide comprising a domain selected from the group consisting of SEQ ID NO:1 and SEQ ID NO:3 to induce MyT1 expression. Still another embodiment of the invention is the use of a polypeptide comprising a domain selected from the group consisting of SEQ ID NO:2 and SEQ ID NO:4 to induce the expression of a member of the SENS family. In an embodiment, the member is Gfi1. Indeed, as is shown in this invention, polypeptides of the atonal group of polypeptides do induce the members of the SENS family. As the domain is conserved over the different species, and can be found in the human atonal homologue Hath1, it is likely to assume that the human SENS homologue Gfi1 is induced too by a polypeptide according to the invention.

A further embodiment of the invention is the use of a polypeptide comprising a domain selected from the group consisting of SEQ ID NO:1, SEQ ID NO:3, SEQ ID NO:5, SEQ ID NO:6 and SEQ ID NO:7 to induce sensory organ precursors in vertebrates. In an embodiment, the vertebrate is a mammal. In another embodiment, the vertebrate is a human.

A further embodiment of the invention is the use of a polypeptide comprising a domain selected from the group consisting of SEQ ID NO:2 and SEQ ID NO:4 to induce vertebrate inner hair cells. In an embodiment, the polypeptide is an artificial polypeptide. N an embodiment, the vertebrate is a mammal. In an embodiment, the vertebrate is a human.

A further embodiment of the invention is the use of a polypeptide according to the invention, or an antibody directed against a domain according to an embodiment of the invention to treat cancer. Indeed, it is know that, in the case of Merkel Cell Carcinoma (MCC), cells that have lost Hath1 expression lose their neuroendocrine phenotype, which results in a very aggressive tumor phenotype (Leonard et al., Int. I. Cancer, 101, 103-110, 2002). Expression of a polypeptide comprising a domain selected from the group consisting of SEQ ID NO:2 and SEQ ID NO:4 in the affected cells will force MCC differentiation and slow tumor progression. On the other hand, it is known that overexpression of Gfi1 is involved in cancers such as T cell lymphoma (Gilks et al., Mol. Cell. Biol. 13, 1759-1768, 1993) and adult T-cell leukemia/lymphoma (ATLL) (Sakai et al., Int. J. Hematol. 73, 507-515, 2001). In the latter case, Gfi1 is not induced by STAT, but may be induced by a protein of the atonal group of polypeptides. In that case, antibodies against a domain with SEQ ID NO:2 or SEQ ID NO:4 can block the atonal-specific induction. Alternatively, overexpression of a polypeptide comprising a domain selected from the group consisting of SEQ ID NO:1, SEQ ID NO:3, SEQ ID NO:5, SEQ ID NO:6 and SEQ ID NO:7 may outcompete interaction by the atonal group of polypeptides and block the atonal-type induction.

Still another embodiment of the invention is a method of treating an animal with a deficiency in cerebellar granule neurons or their precursors comprising delivery of a therapeutically effective amount of a polypeptide comprising a domain selected from the group consisting of SEQ ID NO:2 and SEQ ID NO:4 to a cell of the animal. Another aspect of the invention is a method of promoting mechanoreceptive cell growth in an animal, comprising delivery of a therapeutically effective amount of a polypeptide comprising a domain selected from the group consisting of SEQ ID NO:2 and SEQ ID NO:4 to a cell of the animal. Still another embodiment of the invention is a method of generating inner ear hair cells comprising delivery of a therapeutically effective amount of a polypeptide comprising a domain selected from the group consisting of SEQ ID NO:2 and SEQ ID NO:4 to a cell of the animal. Still another embodiment of the invention is a method of treating an animal for hearing impairment comprising delivery of a therapeutically effective amount of a polypeptide comprising a domain selected from the group consisting of SEQ ID NO:2 and SEQ ID NO:4 to a cell of the animal. In an embodiment, the polypeptide is an artificial polypeptide. In an embodiment, the animal is a mammal. In an example, the animal is a human. An embodiment is a method according to the invention whereby delivery is realized by in situ synthesis of the polypeptide. Such an in situ synthesis can be realized, as a non-limiting example, by delivering the nucleic acid to the cell by gene therapy.

### BRIEF DESCRIPTION OF THE FIGURES

FIG. 1. The evolutionary relationship of Atonal-related polypeptides and their neurogenic capacities. Panel A: A neighbor-joining tree representing three subgroups of Atonal-related polypeptides, NeuroD, Neurogenin and Atonal group polypeptides. Some example sequences of the bHLH domain for Neurogenin and Atonal group polypeptides are shown. The length of lines is not corresponded to the revolutionary distance. The amino acids in red and with red under-line indicate identical and similar sequences between Neurogenin and Atonal group polypeptides respectively. Panels B and C: N-tubulin stained un-injected Xenopus embryos at stage 14 and 19 respectively. Panels D and E: N-tubulin stained Xenopus embryos at stage 14 and 19 respectively, injected with 500 pg NGN1 mRNA into one cell (right side) of two cell-stage embryos. Panels F and G: N-tubulin stained Xenopus embryos at stage 14 and 19 respectively, injected with 500 pg ATO mRNAs into one cell (right side) of two cell-stage embryos.

FIG. 2. NGNs have weak neurogenic capacity in Drosophila. Panel A: Part of a wild-type fly wing showing no sensory bristles along A-P axis (dotted line). Panel B: A UASMath1/+; dppGal4/+fly wing revealing a large numbers of ectopic sensory bristles along A-P axis. Panel C: A UASngn1/+; dppGal4/+fly wing displaying very few ectopic sensory bristles (arrows) along A-P axis. Panel D: Quantitative analysis of the number of ectopic bristles per fly induced by expression of MATH1 or NGN1. Thirty flies were counted for NGN1, and 32 flies were counted for MATH1 (P<0.001). Panel E: A UASngn1/UASngn2; dppGal4/+fly wing showing a similar number of bristles (arrows) as flies expressing NGN1 alone. Panel F: A UASngn1/UASMath3; dppGal4/+fly wing presenting a similar number of bristles (arrows) as flies expressing NGN1 alone.

FIG. 3. NGN1 fails to induce SOP formation in Drosophila. Panel A: The normal pattern of SOPs in a third instar larval (L3) wing disc from an A101-LacZ fly revealed by anti-β-GAL (green). Panel B: The pattern of mis-expressed ATO in L3 of UASato/+; dppGal4/A101 fly wing disc, stained by anti-ATO (red). Panel C: The pattern of SOPs in L3 of UASato/+; dppGal4/A101 fly wing disc, stained by anti-β-GAL (green). Panel D: A merged image of Panels B and C shows that mis-expression of ATO along the A-P axis causes ectopic SOP formation. Panel E: A merged image of double stained (anti-MATH1 in red, anti-β-GAL in green) L3 of UASMath1/+; dppGal4/A101 fly wing disc showing that mis-expression of MATH1 along the A-P axis causes ectopic SOP formation. Panel F: The pattern of mis-expressed NGN1 in L3 of UASngn1, dppGal4/A101 fly wing disc, stained by anti-NGN1 (red). Panel G: The pattern of SOPs in L3 of UASngn1, dppGal4/A101 fly wing disc, stained by anti-β-GAL (green). Panel H: A merged image of Panels F and G reveals no detectable ectopic SOPs forming by mis-expression of NGN1.

FIG. 4. NGN1 interacts with Da and the Notch signaling pathway in Drosophila. Panel A: Autoradiograph of SDS-PAGE gels from co-immunoprecipitation using anti-Myc antibodies of ³⁵S-labeled ATO, MATH1 and NGN1 in the presence (the first three lanes) and absence (the last lane) of Myc tagged Da. Panel B: A UASngn1, dppGal4/TM6 fly wing revealing a few ectopic sensory bristles along A-P axis. Panel C: A da/+; UASngn1, dppGal4/+fly (Da^{+/-}) wing showing no ectopic sensory bristles along A-P axis. Panel D: A N⁸/+; UASngn1, dppGal4/+fly (Notch^{+/-}) wing displaying an increased number of bristles along A-P axis. Panel E: A UASN^{intra}/+; UASngn1, dppGal4/+fly (expressing constitutively active form of Notch) wing presenting a complete inhibition of ectopic sensory bristles induction by NGN1. Panel F: A UASm8/UASngn1, dppGal4/+ fly wing showing no ectopic sensory bristles along A-P axis. Panel G: Quantitative analysis of the number of ectopic bristles per fly induced by expressing NGN1 alone in Da^{+/-}, Notch^{+/-} background, or with constitutively active Notch, or the members of enhancer split complex, m8 or mδ. Forty-five flies were counted (P<0.001) per assay.

FIG. 5. NGN1 fails to induce SENS expression. Panel A: The expression pattern of SENS in L3 of wild-type (cs) fly wing disc, stained with anti-SENS (green). Panel B: A L3 of UASato/+; dppGal4/+fly wing disc, double stained with anti-ATO (red) and anti-SENS (green) reveals that mis-expression of ATO induces SENS expressions. Panel C: A L3 of UASMath1/+; dppGal4/+fly wing disc, double stained with anti-MATH1 (red) and anti-SENS (green) displaces that mis-expression of MATH1 induces SENS expressions. Panel D: The expression pattern of NGN1 in L3 of UASngn1, dppGal4/TM6 fly wing disc, stained with anti-NGN1 (red). Panel E: The expression pattern of SENS in L3 of UASngn1, dppGal4/TM6 fly wing disc, stained with anti-SENS (green). Panel F: A merged image of Panels D and E shows that no detectable ectopic expression of SENS induced by mis-expression of NGN1.

FIG. 6. NGN1 does not synergize with SENS. Panel A: A scutellum of UASsens/+; C5Gal4/+ fly. Ectopic bristles indicated by arrows (wild-type fly has four large bristles on scutellum). Panel B: Ectopic bristles (some indicated by arrows) on UASsens/+; C5Gal4/UASngn1 fly scutellum. Panel C: Ectopic bristles (some indicated by arrows) on UASMath1/+; C5Gal4/+fly scutellum. Panel D: A scutellum of UASsens/+; UASMath1/+; C5Gal4/+fly. A large numbers of ectopic bristles, induced by co-expressing MATH1 and SENS, indicates a strong synergy between NGN1 and SENS. Panel E: Quantitative comparison of proneural activity between SENS^{+/-} (UASngn1, dppGal4/sens or UASMath1/+; dppGal4/sens) and SENS^{+/+} (UASngn1, dppGal4/TM6 or UASMath1/+; dppGal4/TM6) background for NGN1 and MATH1. The number of ectopic bristles is decreased by 40% in a SENS^{+/-} background. The proneural activity is assayed by counting the number of sensory bristles induced by NGN1 or MATH1 with dppGal4 driver. Fifty flies were examined per assay. P<0.001 for MATH1. From Panels F to I are the N-tubulin stained Xenopus embryos at stage 14. The embryos were injected or co-injected with different mRNA into one cell (right side) of two cell-stage embryos. Panel F: An embryo, injected with X-MyT1, causes an increase in the number of neurons. Panel G: An embryo, injected with NGN1, shows ectopic neuron induction at the injection side. Panel H: An embryo, co-injected with X-MyT1 and NGN1, causes a dramatic increased neuron induction. Panel I: An embryo, co-injected with X-Myt1 and ATO, does not showing a detectable increase in neuron formation.

FIG. 7. Changing three non-DNA binding amino acids to ATO in basic domain transfers NGN1 into a potent proneural polypeptide in Drosophila. Panel A: The amino acid sequence of the basic domains of ATO (red) and NGN1 (purple). The group-specific amino acids are in green. Panel B: A schematic representation of NGNbATO. The exchanged group-specific amino acids are in red. Panel C: Quantitative analysis of proneural activity of mis-expressed ATO, NGN1 and NGNbATO showing that in contrast of NGN1, mis-expression of NGNbATO induces a similar amount of bristles along A-P axis as mis-expression of ATO, but the number of bristles are reduced by 45% in a SENS+/−background. The insets show a wing disc (upper) and a part of a wing (lower) of a UASNGNbATO/+; dppGAL4/+animal. Double staining with anti-NGN1 (red) and anti-SENS (green) demonstrates that mis-expression of NGNbATO causes ectopic expressions of SENS and induces ectopic bristles along A-P axis. Panel D: A schematic representation of ATObNGN. The exchanged group-specific amino acids are in green. Panel E: N-tubulin stained Xenopus embryos at stage 19, injected with 500 pg of ATO. Panel F: N-tubulin stained Xenopus embryos at stage 14, injected with 500 pg of ATObNGN. Panel G: N-tubulin stained Xenopus embryos at stage 14, injected with 100 pg of ATObNGN mRNA into one cell (right side) of two cell-stage embryos. Panel H: N-tubulin stained Xenopus embryos at stage 19, injected with 100 pg of ATObNGN. Panel I: N-tubulin stained Xenopus embryos at stage 14, injected with 100 pg of ATObNGN and 250 pg mRNA X-MyT1. Panel J: N-tubulin stained Xenopus embryos at stage 19, co-injected with 100 pg of ATObNGN and 250 pg X-MyT1.

FIG. 8. A novel motif in Helix 2 mediates NGN but not ATO proneural activity. Panel A: The amino acid sequence of the HLH domains of ATO and NGN1 group proteins. The group-specific amino acids in Helix2 are highlighted. Panel B: Schematic representation of NGN^{H2ATO} and ATO^{H2NGN}. Panel C: Quantitative analysis of proneural activity of mis-expressed ATO (n=30), NGN1 (n=45), NGN^{H2ATO} (n=30), where “n” represents the numbers of flies examined. The proneural activity is assayed by counting the number of sensory bristles along A-P axis induced by these proteins with the dppGal4 driver. Panel D: N-tubulin stained Xenopus embryo at stage 19, injected with 100 pg of ATO^{H2NGN} mRNA into one cell (right side) of two cell-stage embryos. Panel E: N-tubulin stained Xenopus embryo at stage 19, co-injected with 100 pg of ATO^{H2NGN} and 250 pg X-MyT1 mRNA. Panels F and G: A 3-D structural model of the bHLH domain based on the crystal structure of the MyoD protein showing the exchanged residues: three residues (yellow) in basic domain (Panel F) and five in the Helix2 domain (Panel G).

FIG. 9. Loss of Hath1 expression induces an aggressive behavior in Merkel Carcinoma cells. Doubling times of the Hath1-expressing Merkel Carcinoma cell lines MCC1 and MCC6, in comparison with the cell lines MCC13, MCC14/2 and MCC26#7 that have lost Hath1 expression.

### DETAILED DESCRIPTION OF THE INVENTION

Definitions

The following definitions are set forth to illustrate and define the meaning and scope of various terms used to describe the invention herein.

A “biological active artificial polypeptide” means any polypeptide that is not naturally occurring. It includes, but is not limited to mutants, deleted and/or truncated polypeptides, fusion polypeptides, modified polypeptides and peptido-mimetics. Biological active as used here means that the protein can be used to specify the neuronal lineage identity of stem cells.

The terms “protein” and “polypeptide” as used in this application are interchangeable. “Polypeptide” refers to a polymer of amino acids and does not refer to a specific length of the molecule. This term also includes post-translational modifications of the polypeptide, such as glycosylation, phosphorylation and acetylation.

The “biological activity of a domain,” as used here, means the specific induction of neuronal precursor cells as measured either in Xenopus (for polypeptides comprising the domain consisting of SEQ ID NO: 1 or SEQ ID NO:3) or in Drosophila (for polypeptides comprising the domain consisting of SEQ ID NO:2 or SEQ ID NO:4). Alternatively, the biological activity may be measured as induction of MyT1 messenger RNA in Xenopus cells (for polypeptides comprising the domain consisting of SEQ ID NO:1 or SEQ ID NO:3) of as the induction of SENS mRNA in Drosophila (for polypeptides comprising the domain consisting of SEQ ID NO:2 or SEQ ID NO:4).

An “active domain” is a domain that shows biological activity in the cells used; an “inactive domain” is a domain that shows a biological activity that is less than 50% of the activity of that of the active domain when used in the same cells. In an embodiment, the biological activity of the inactive domain is even less than 10% than that of the active domain. Note that an active domain can be an inactive one and that an inactive domain can be an active one when both domains are tested in another cell type.

A “polypeptide of the atonal group” as used here means a polypeptide comprising a domain selected from the group consisting of SEQ ID NO:2 and SEQ ID NO:4; “atonal-type induction” is the induction that is obtained by expression of such a polypeptide.

The “SENS family,” as used here, consists of polypeptides that are structural and functional homologous to the Drosophila senseless protein. It includes, but is not limited to the human Gfi-1 protein and the C. elegans PAG-3 protein.

“Delivery of a polypeptide into a cell” may be direct, e.g., by microinjection or by uptake by the cell, or it may be indirect, by transfer of a nucleic acid encoding the polypeptide into the cell. In the latter case, the expression of the polypeptide may be transient, or it may be stable expressed, and the nucleic acid may be integrated in the genome.

A “therapeutically effective amount” as used here is defined as the amount required to obtain a significant improvement of some symptom associated with the disease treated.

“Compound” means any chemical of biological compound, including simple or complex organic and inorganic molecules, peptides, peptido-mimetics, proteins, antibodies, carbohydrates, nucleic acids or derivatives thereof.

### EXAMPLES

Materials and Methods to the Examples

DNA Construction and Microinjection Procedures

The full-length coding region of NGN 1 and cDNA of ATO were cloned into the pCS2MT (Tumer and Weintraub, 1994) vector in the EcoRI-XbaI site and SnaBI site from their original cDNA Bluescript plasmid (pBS). Plasmid DNA containing a coding region of MyT1 in the pCS2MT was provided by Crist. The exchanging version of open reading frame (ORF) of NGN1 and cDNA of ATO (changed three amino acids in basic domain from NGN1 to ATO, termed NGN^{bATO} and from ATO to NGN1, termed ATO^{bNGN}) were obtained by site-directed mutagenetic PCR amplification from NGN1-pBS and ATO-pBS plasmid, and cloned into EcoRI-HindIII and KpnI sites of the pBS. NGN^{bATO}-pBS was cut by XbaI-KpnI and recloned into pUAST vector. ATO^{bNGN}-pBS was cut by EcoRI and recloned into pCS2MT.

All mRNAs (constructs in pCS2MT) were transcribed using SP6 RNA polymerase as described (Kintner and Melton, 1987), and were injected in a volume of 5 nl at a concentration of 50 to 100 pg/nl into a single blastomere of Xenopus embryos at the two-cell stage as described previously (Coffman et al., 1990). Embryos were collected at stage 14 or 19. Whole-mount in situ hybridization was performed as described (Chitnis et al., 1995). Preparation of N-tubilin probe was as described previously (Oschwald et al., 1991; Chitnis et al., 1995). Transgenic fly lines containing UAS-NGN^{bATO} insertion in different chromosomes were generated by injecting NGN^{bATO} pUAST plasmid DNA into fly embryos, and selecting upon eye color.

Plasmid Construction and Microinjection for ngn^{H2ato} and ato^{H2ngn}

The ato cDNA was subcloned into pCS2+vector (Rupp et al., 1994) using the SnaBI site, hence creating pCS2+ato. The full-length coding region of NGN1 was subcloned into the EcoRI-XhoI sites of the pCS2+vector, resulting in pCS2+ngn. The pCS2+X-MyT1 plasmid was described earlier (Bellefroid et al., 1996). DNA coding for ngn^{H2ato} and ato^{H2ngn} were obtained by site-directed mutagenesis PCR amplification from ngn1-pBS and ato-pBS plasmids. The ngn H² at fragment was cloned into the XbaI-KpnI sites of pUAST vector. The ato^{H2ngn} fragment was cloned into the EcoRI site of pCS2+vector. The cDNA templates were linearized for in vitro transcription and capped mRNAs were generated using SP6 RNA Polymerase (Promega). mRNAs were injected in a volume of 5 nl at a concentration of 20 to 200 pg/nl, into a single blastomere of Xenopus laevis embryos at the two-cell stage. The injected side in the picture shown is always on the right of the embryo. During injection, embryos were kept as described (Vleminckx et al., 1997) and collected at stages 15 and 19. Staging was according to Nieuwkoop and Faber (Nieuwkoop and Faber, 1994). The embryos were fixed in 1×MEMFA (0.1 M MOPS, pH 7.4, 2 mM EGTA, 1 mM MgSO₄, 3.7% formaldehyde) for one to two hours at room temperature.

Fly Stocks

Most mutant fly strains used in this study have been published and are described and referenced throughout the text. N⁸, Da and SENS mutant stocks and flies containing UASm8, UASmδ were obtained from the Bloomington Stock Centre. Flies were raised on standard fly food. All crosses involving mutant stocks were performed at 25° C.

Transgenic fly lines containing UAS-NGN^{bATO} insertion in different chromosomes were generated by injecting NGN^{bATO}-pUAST plasmid DNA into fly embryos, and selecting upon eye color.

In Situ Hybridization

Whole mount in situ hybridization was performed as described (Harland, 1991) using a digoxigenin-labeled antisense N-tubulin probe. Preparation of N-tubulin probe was as described previously (Chitnis et al., 1995; Oschwald et al., 1991). Detection was performed using the BM Purple AP substrate (Roche Molecular Biochemicals). When staining was complete, the embryos were rinsed in PTW (1×PBS, 0.1% Tween-20) and re-fixed overnight in Bouin's fix (9% formaldehyde, 5% glacial acetic acid and 1% picric acid saturated in distilled water). To remove any chromogenic or residual components, the embryos were subjected to several washes of 70% ethanol/30% PTW, before bleaching them in a solution containing 1% H₂O₂, 5% formamide and 0.5×SSC (Mayor et al., 1995). For each injection, at least 50 embryos were examined per se.

Immunohistochemistry

Third instar larval wing discs were dissected in PBT and fixed with 4% formaldehyde in PBT for 15 minutes. After five minutes of five times washing with PBT, and one hour blocking in 1×PAXDG buffer (PBT with 5% normal goat serum, 1% bovine serum albumin, 0.1% deoxycholate, and 1% Triton X-100) (Mardon et al., 1994), the wing discs were incubated with anti-β-Gal (Promega, 1:2000), anti-ATO (1:1000), anti-NGN1 (1:100), anti-Math1 (1:100) anti-SENS (1:250) in 1×PAXDG. Samples were washed 15 minutes with PBT for five times and incubated with the appropriate secondary antibodies (1:250) in 1×PAXDG. After five times 30-minute washes with PBT, wing discs were mounted in Vectashield mounting medium (vector) and detected using confocal microscopy (BioRad 1024). Adult fly wings and scutella were mounted in 70% ethanol and documented using Leica microscopes and software.

Evolutionary Trace Analysis

A multiple sequence alignment and a sequence identity tree were generated using the pairwise sequence comparisons algorithm PILEUP (Feng and Doolittle, 1987) from the GCG sequence analysis package (Devereux et al., 1984). There were no gaps in the alignment when using default values and only one gap when we also considered the sequence of the crystal structure. The Evolutionary Trace was performed as described previously (Lichtarge et al., 1996b).

### Example 1

### Drosophila and Xenopus Use Different Group of Proneural Polypeptides for SOP Selection in the PNS

The Vertebrate Ectoderm Responds to NGN Group Polypeptides

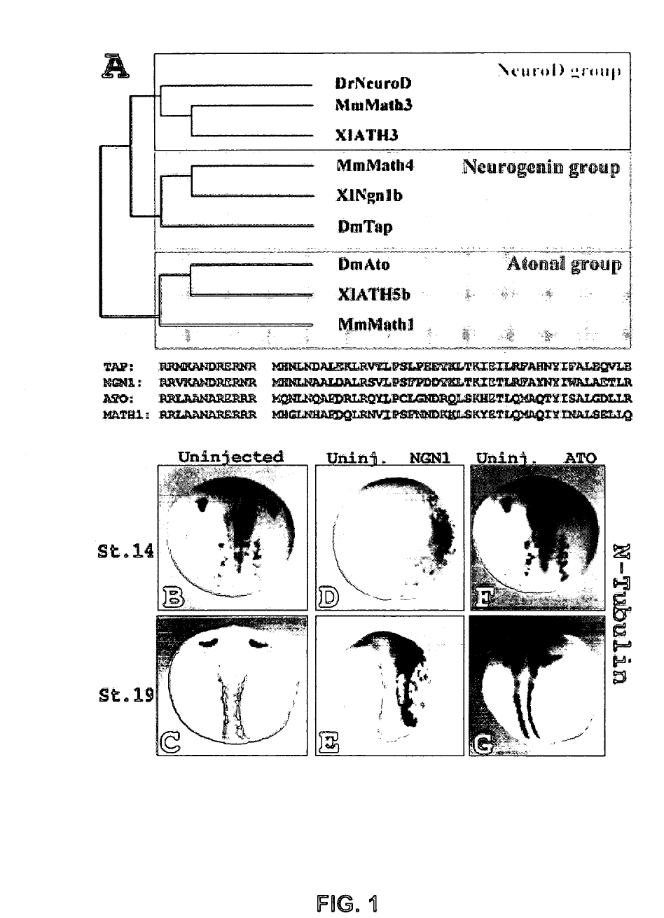

To assay the proneural activity of mouse NGN1 and fly ATO, the mRNAs of proneural genes were injected into one cell of two-cell stage Xenopus embryos, and neuronal induction was detected by staining for N-tubulin at stage 14 and 19. Compared to uninjected embryos (FIG. 1, Panels B and C), injection of NGN1 mRNA causes a dramatic increase in the number of neurons detected by N-tubulin expression (FIG. 1, Panels D and E). In contrast, injection of ATO mRNA does not cause a detectable increase in neuron formation (FIG. 1, Panels F and G, right side). These data suggest that the vertebrate ectoderm responds specifically to NGN group polypeptides but not ATO group polypeptides to induce neurogenesis. The Drosophila ectoderm responds to ATO group polypeptides

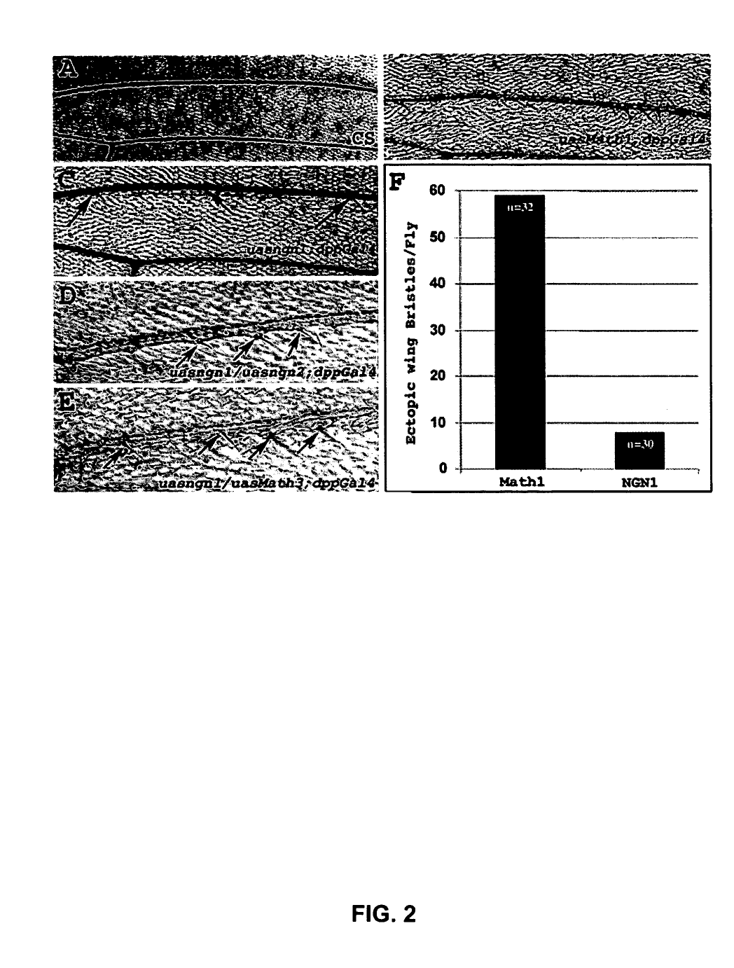

One possibility is that NGNs are more potent neural inducers than ATOs and stronger neuronal induction is needed in vertebrate ectoderm than in the Drosophila ectoderm. To test this, we mis-expressed ATOs and NGNs in Drosophila using the UAS/Gal4 system and assayed neural induction by counting the number of sensory bristles produced on the wing. More than 20 independent transgenic lines were generated for UASNGN1 and UASNGN2. None of the NGN2 lines showed any neural induction with five different wing Gal4 drivers. Sixteen out of 23 NGN1 transgenic lines showed no neural induction. The other seven showed very weak induction (see below) with only two of the wing Gal4 drivers, dppGal4 and ap-Gal4. Therefore, combination of dppGal4 and the strongest UASNGN1 transgenic line were used in the remainder of this study. The dppGal4 driver in Drosophila is used to induce genes of interest along the anterior-posterior (A-P) axis of the wing disc. Wild-type flies have no sensory bristles on the A-P axis of the wing (FIG. 2, Panel A). A large number of sensory bristles are found along the A-P axis of the wing with 100% penetrance when MATH1 (FIG. 2, Panel B) or ATO (data not shown) is mis-expressed. However, expression of NGN1 (with the strongest transgenic line) results in the appearance of very few bristles (indicated by arrows) in about 70% of the flies (FIG. 2, Panel C). Quantitative analysis reveals that the number of sensory bristles induced by MATH1 is more than six-fold the number induced by NGN1 (FIG. 2, Panel D).

Since NGN1 and NGN2 are often co-expressed in the vertebrate PNS, we therefore tested whether their co-expression is required for neuronal induction. Our result shows that co-expression of NGN1 and NGN2 gives the same effect as expression of NGN1 alone (FIG. 2, Panel E). Although, it has been shown that NeuroD group polypeptides have no proneural activity, they seem to be direct targets of NGN polypeptides. Therefore, it is possible that the weak neuronal induction of mouse NGN1 is due to the lack of homologues of NeuroD polypeptides in flies. However, co-expression of NGN1 and MATH3, a NeuroD group member, has no effect on the proneural activity of NGN1 (FIG. 2, Panel F).

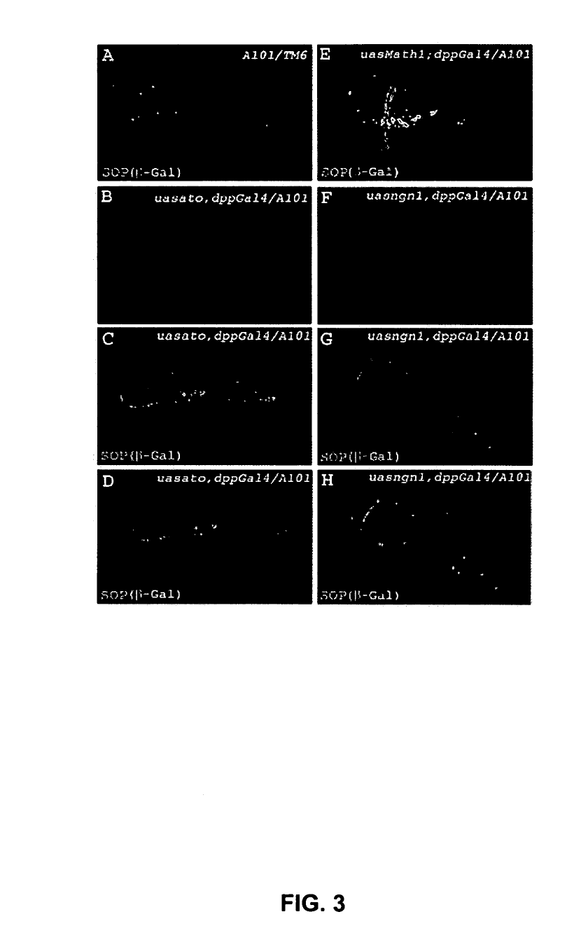

Still, it is possible that NGN1 is able to induce SOPs, but most of these SOPs fail to differentiate properly in order to give rise to sensory organs. Therefore, we examined SOP formation directly by expressing NGN1, ATO and MATH1 with dppGal4 in the A101 flies, which carry an SOP-specific LacZ enhancer trap. The normal pattern of SOPs is revealed by anti-β-GAL staining in A101-expressing wing discs (FIG. 3, Panel A). Mis-expression of ATO (FIG. 3, Panel B) along A-P axis of the wing disc results in the induction of ectopic SOPs (FIG. 3, Panel C) within the domain of ATO expression (FIG. 3, Panel D). Expression of MATH1 (FIG. 3, Panel E) has a similar result as ATO. In contrast, no detectable formation of ectopic SOPs (FIG. 3, Panels G and H) is found by expression of NGN1 (FIG. 3, Panels F and H). These data suggest that the fly ectoderm takes expression of ATOs but not NGNs as a signal for SOP formation.

The Xenopus and Drosophila data together indicate that ATO and NGN polypeptides use different mechanisms to specify SOPs in Drosophila and vertebrates PNS respectively.

Mouse NGN1 Can Interact Both In Vitro and In Vivo with Fly Daughterless in Drosophila

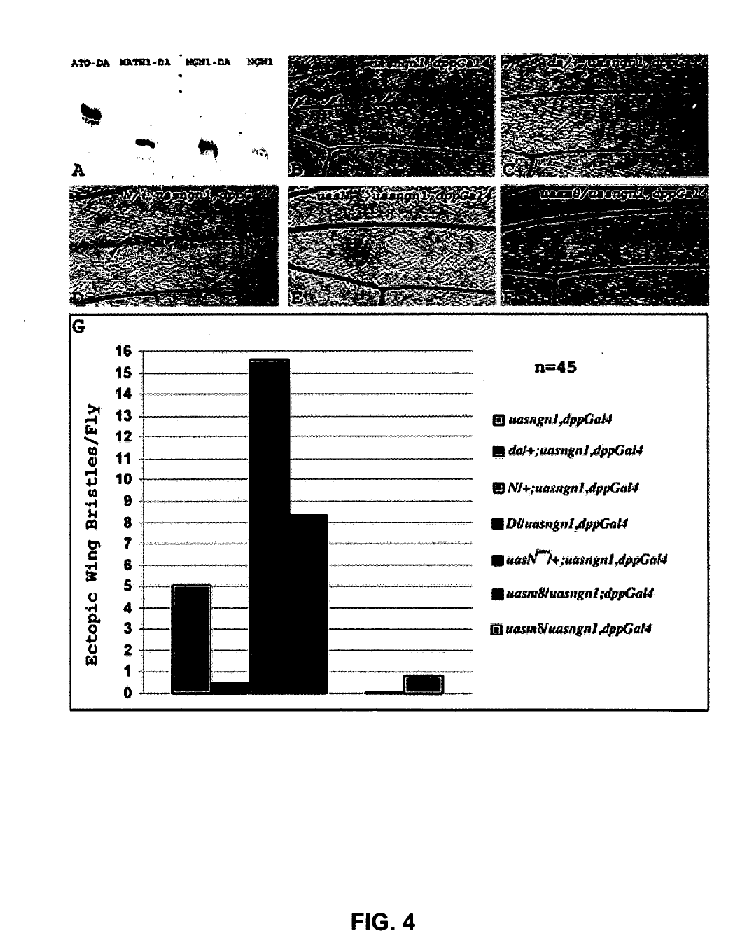

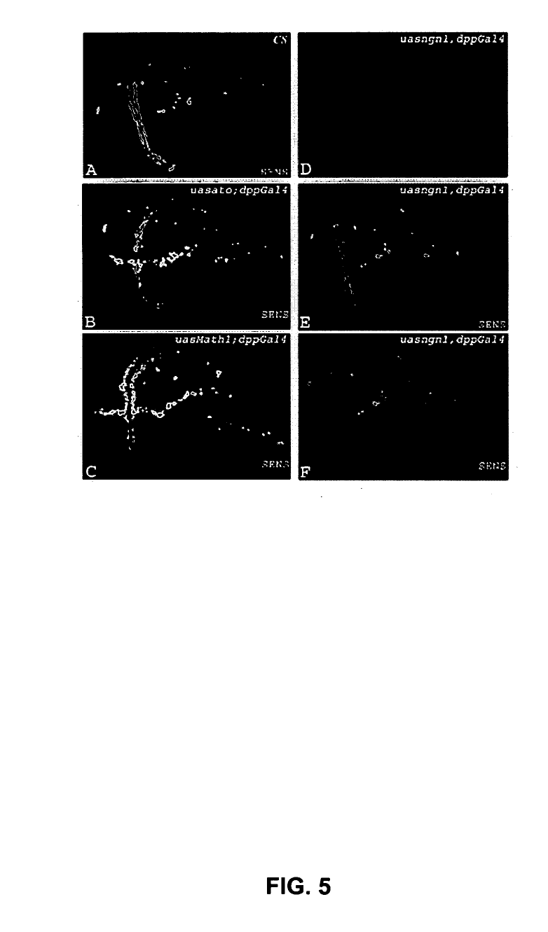

One explanation for the failure of NGN1 to induce neurogenesis is that NGN1 is unable to form heterodimers with fly Daughterless (Da). In order to test whether mouse NGN1 can form heterodimers with fly daughterless, co-IP experiment was performed, in which S³⁵-labeled ATO, MATH1 or NGN1 was co-precipitated with Da-Myc using anti-Muc antibodies (FIG. 4, Panel A). The precipitates (heterodimers of proneural polypeptides and Da-Myc) were run on SDS-PAGE gel, dried and detected by autoradiography. No NGN1 can be detected after precipitation in the absence of Da. In the presence of Da, just as mouse MATH1 or fly ATO, mouse NGN1 can be co-precipitated. These results suggest that mouse NGN1 can bind physically to fly Daughterless in vitro. To test if they interact with each other in vivo, flies containing a UASngn1 insertion driven by dppGAL4 were crossed with Da mutant flies. The number of sensory bristles produced by NGN1 along A-P axis is decreased in a heterozygous Da background (Da^{+/-}, FIG. 4, Panels C and G) compared to a wild-type background (FIG. 4, Panels B and G). Therefore, mouse NGN1 can physically and genetically interact with fly Daughterless in Drosophila in a dosage-sensitive manner.

Mouse NGN1 Can be Regulated by the Fly Notch Signaling Pathway in Drosophila

It is also possible that mouse NGN1 cannot interact with the Drosophila Notch signaling pathway. To test it, we examined ectopic neural induction of NGN1 in absence of one copy of Notch (N^{+/-}) or with co-expression of a constitutively active form of Notch (N^{intra}). The results show that the proneural activity of NGN1 is strongly enhanced in a N^{+/-} background (FIG. 4, Panel D) and totally inhibited in a N^{intra} background (FIG. 4, Panel E). Similarly, co-expression of the members of the enhancer split complex, m8 (FIG. 4, Panel F) and mδ (data not shown) inhibits the proneural activity of NGN1. (Only one or two bristles can be found in less than 10% of flies.) These results are quantified in FIG. 4, Panel G. The number of bristles induced by NGN1 decreases ten-fold in a Da^{+/-} background. The average of number of bristles induced by NGN1 increase from five to more than 15 per fly in a N^{+/-} background. The average number of bristles induced by NGN1 decrease from five to less than one by co-expression of N^{intra} or member of the enhancer split complex m8 and mδ. These data indicate that mouse NGN1 can be regulated by the fly Notch signaling pathway in Drosophila. However, they also suggest that the principle reason for NGN1's weak proneural activity is its inability to efficiently repress Notch signaling when it is overexpressed.

### Example 2

### ATOs and NGNs Interact with Different Zn Finger Polypeptides During Sop Specification

ATO but not NGN1 Induces SENS

SOP formation in Drosophila requires the Zn finger protein Senseless (SENS). Fly proneural polypeptides first induce senseless expression and then synergize with it in a positive feedback loop. This enhances the ability of proneural genes to down-regulate Notch signaling in the presumptive SOP and results in SOP selection. In vertebrates, Senseless-like proteins have not been shown to act in SOP formation. To test the possibility that SENS represents a divergence point in the mechanism of SOP selection, we compared the ability of two group proneural polypeptides for regulating and interacting with SENS.

First, we examined the SENS expression pattern in wing discs, where the proneural polypeptides NGN1, MATH1 or ATO were mis-expressed. The expression of SENS is detected with anti-SENS (green), and proneural polypeptides were stained with their respective antibodies (red). SENS expression in wild-type fly wing disc (FIG. 5, Panel A) prefigures SOP formation. Ectopic SENS expression is detected along A-P axis of wing disc when ATO (FIG. 5, Panel B) or MATH1 (FIG. 5, Panel C) are mis-expressed. However, no ectopic SENS expression (FIG. 5, Panels E and F) can be detected when NGN1 (FIG. 5, Panels D and F) is mis-expressed. These data suggest that NGN1 does not induce SENS expression, whereas ATO and Math1 can induce SENS.

ATO but not NGN1 Interacts with SENS

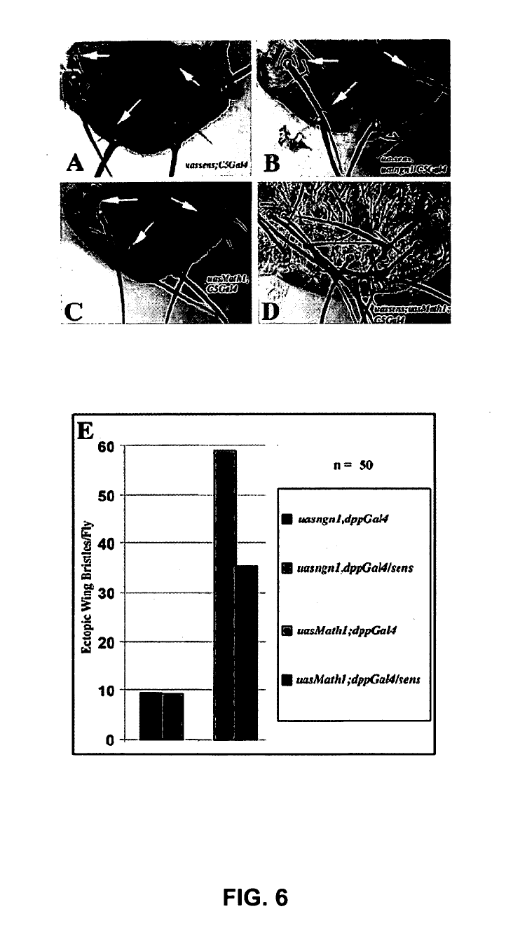

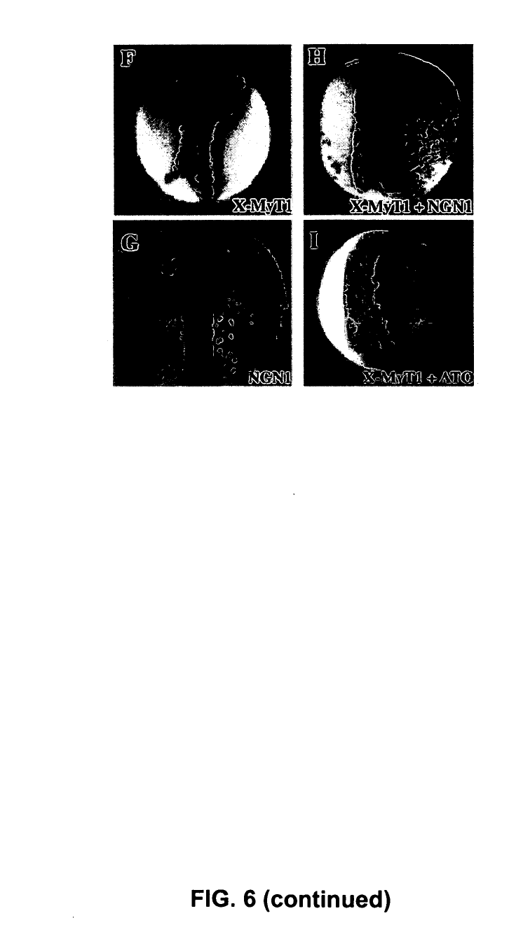

Although, NGN1 does not induce SENS, it is possible that NGN1 can synergize with SENS if the requirement to induce SENS expression is bypassed. We, therefore, compared the ability of NGN1 and MATH1 to synergize to SENS in vivo by co-expressing NGN1 or MATH1 with SENS, using C5Gal4 (FIG. 6) or dppGal4 (data not shown). Neural induction was examined by counting the ectopic bristles induced on scutellums. Expression of SENS (FIG. 6, Panel A) or MATH1 (FIG. 6, Panel C) alone cause a number of ectopic sensory bristles on scutellum. No ectopic sensory bristles on scutellum have been found when NGN1 was expressed alone (data not shown). Co-expression of NGN1 and SENS has the same effect on the scutellum as the mis-expressing SENS alone (FIG. 6, Panel B). Co-expression of MATH1 and SENS cause appearance of a large number of bristles on scutellum. Very similar data were obtained using the dppGal4 driver.

These data suggest that NGN1 does not synergize with SENS, thus explaining its weak proneural activity. To test if SENS plays any role in NGN1's activity, flies, mis-expressing NGN1 or MATH1 were crossed with SENS mutant flies. The average number of sensory bristles produced by MATH1 along A-P axis is reduced by 42% if a single copy of SENS is removed (SENS^{+/-}, FIG. 6, Panel E) suggesting dosage-sensitive interactions. In contrast, no effect on NGN1 activity in a SENS^{+/-} background was observed (FIG. 6, Panel E). These data indicate that NGN1 does not interact with SENS, therefore, SENS is one extrinsic difference between ATO and NGN polypeptides and, therefore, between SOP formation in flies and vertebrates.

NGN1 Interacts with MyT1 to Initiate SOP Formation

It has been shown that the Zn finger protein MyT1 participates in proneural activity in vertebrates and can synergize with NGN polypeptides. In order to test if MyT1 plays a role similar to SENS in vertebrates in the process of SOPs specification, we compared its ability to interact with NGNs and ATOs in Xenopus. MyT1 was injected alone or co-injected with either NGN1 or ATO. As expected, the injection of NGN1 (FIG. 6, Panel F) or MyT1 (FIG. 6, Panel G) mRNA alone in the right side blastomere causes ectopic neural induction. Co-injection of NGN1 and MyT1 mRNAs causes very strong ectopic neuron induction (FIG. 6, Panel H). In contrast, co-injection of ATO and MyT1 mRNA does not cause a detectable increase in neural formation compared to injection of MyT1 mRNA alone (FIG. 6, Panel I). Taken together, all these data suggest that ATO polypeptides synergize with SENS for SOPs formation in Drosophila, whereas NGN polypeptides synergize with MyT1 for SOPs formation in Xenopus, which reflects extrinsic differences in evolutionary divergence of the mechanisms regulating neural precursor selection.

Three Non-DNA Binding Amino Acids in the Basic Domain are the Intrinsic Difference Between NGNs and ATOs

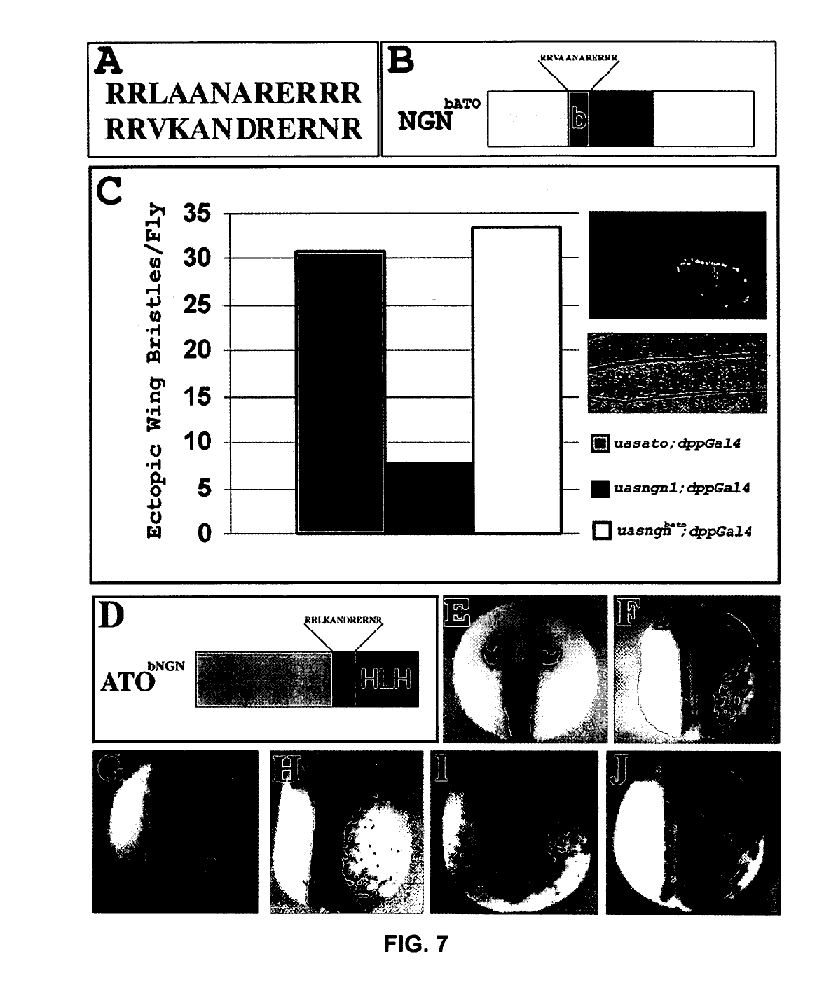

To address the question whether these differential activities and regulatory interactions of NGNs and ATOs can be understood at the level of the proneural proteins themselves, we turned our attention to the comparative analysis of the amino acid sequence of the bHLH domain. Hassan and Bellen (2000) have shown that of the twelve amino acids in the DNA binding basic domain, ATOs and NGNs share eight residues. One residue is variable, and three residues show group specificity: they are highly conserved within each group but are never the same between the two groups (FIG. 7, Panel A, green). Interestingly, the three amino acids form a continuous domain pointing away from the DNA (FIG. 7, Panel B, green). Does this sequence specificity explain the functional difference between ATOs and NGNs? We addressed this question by creating two chimeric proteins exchanging the three group-specific amino acids in the basic domain of NGN1 to those present in ATO (named NGN^{bATO}, FIG. 7, Panel C), or in a reverse way, from ATO to NGN (named ATO^{bNGN}, FIG. 7, Panel D). Mis-expression of NGN^{bATO} causes a large number of bristles along A-P axis of the wings (FIG. 7, Panel E) and results in the ectopic expressions of SENS (FIG. 7, Panel F). Quantitative analysis (FIG. 7, Panel G) shows that like ATO itself, mis-expression of NGNbATO cause an average of about 33 bristles along A-P axis per fly as compared to seven for NGN1. Ectopic neural induction of NGN^{bATO} in absence of one copy of SENS is reduced by 45%. A large number of bristles were induced on scutellum by co-expression of NGN^{bATO} and SENS using the dppGal4 drive (data not shown). These data suggest that the NGN^{bATO} mutant recovers the proneural function in Drosophila. Similarly, injection of ATObNGN mRNA in the right side blastomere causes ectopic neuron induction (FIG. 7, Panel J), which is similar to injection of NGN1 (FIG. 7, Panel H) but is in contrast of ATO injection (FIG. 7, Panel I). Just like NGN1, co-injection of ATO^{bNGN} and MyT1 mRNAs causes very strong ectopic neuron induction (FIG. 7, Panel K). Our results indicate that the group-specific residues in basic domain are the key intrinsic difference between ATO and NGN polypeptides for SOP formation in Drosophila and Xenopus.

### Example 3

### Five Helix2 Residues are Required for Proneural Activity of NGNs but not for ATOs

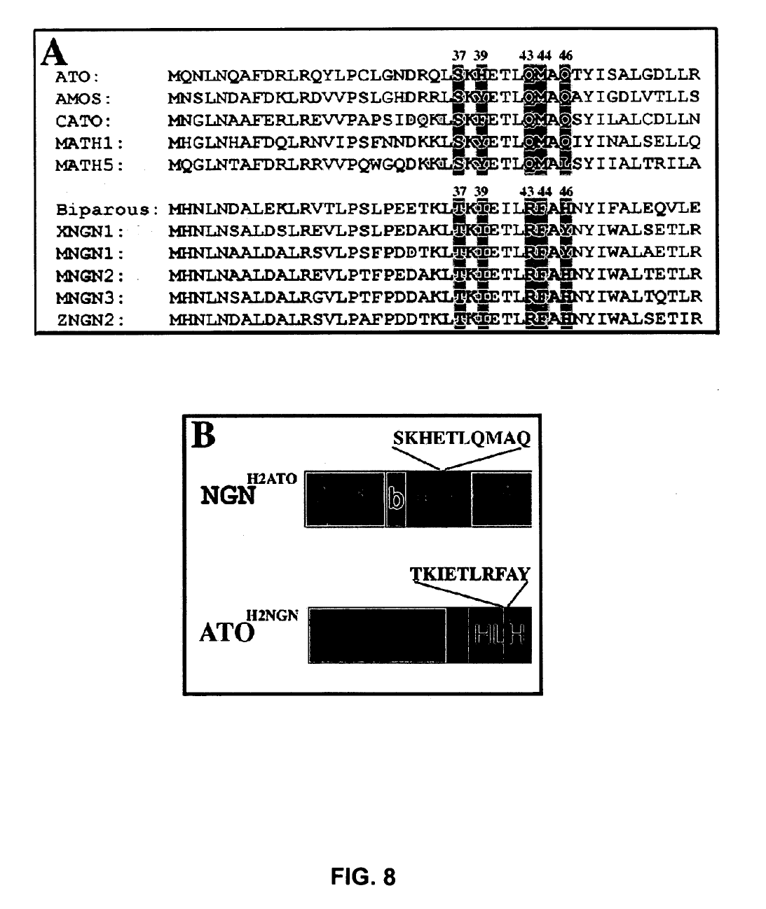

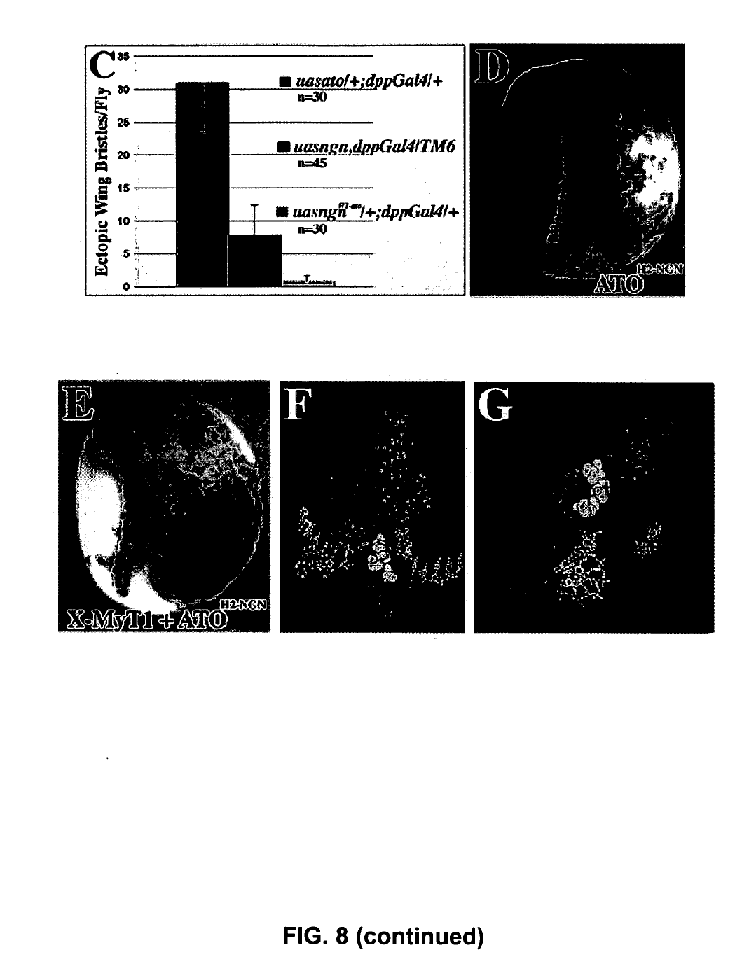

To address the question of whether similar motifs exist in HLH domain of NGN1 and ATO, we compared the amino acid sequence of their HLH domains. We found a number of suggestive amino acids, including an eleven amino acid stretch (36 to 46) within Helix2 in which NGNs and ATOs share six residues. The other five residues (37, 39, 43, 44 and 46) show almost absolute group specificity (FIG. 8, Panel A, highlight). To determine whether these residues are also likely to reflect functional specificity within the ARP family, we turned to the Evolutionary Trace (ET) analysis method which tracks residues whose mutations are associated with functional changes during evolution. This approach has been used to identify novel functional surfaces subsequently confirmed experimentally (Lichtarge et al., 1996a; Lichtarge et al., 1996b; Onrust et al., 1997; Sowa et al., 2001; Sowa et al., 2000), and it has recently been shown to be widely applicable in proteins (Madabushi et al., 2002). In practice, ET relies on a protein family's phylogenetic tree to approximate functional branches. It then successively divides and subdivides a multiple sequence alignment into groups and subgroups that correspond to successive branches of the tree. Each time, ET identifies residue positions of the alignment that are invariant within branches but variable between them (these positions are called class specific). The smallest number of branches at which a position first becomes class specific defines its rank. The top-ranked positions (1) do not vary. Very highly ranked positions (2, 3, etc . . . ) are such that they vary little, and whenever they do, there is also a major evolutionary divergence. In contrast, poorly ranked positions vary more often and their variations occur between closely related species. Thus, top-ranked positions tend to be functionally important, while poorly ranked ones tend not to be. ET identified a number of positions that are jointly important in different bHLH domains, yet that undergo significant variation between them. These residues varied in rank from two to seven, suggesting that they can undergo non-conservative mutations likely to correspond to functional divergence events. These positions tend to be most conserved between NeuroDs and NGNs and then undergo variations in ATOs. This suggests that they may be important for an activity shared by NGNs and NeuroDs, but absent in ATOs. The ability to induce neural precursor cells in vertebrates is precisely such a function. To further investigate the role of these group-specific residues in the functional specificity of NGNs, a chimeric protein, named NGN^{H2ATO} exchanging the group-specific amino acids 37, 39, 43, 44 and 46 in Helix2 of NGN1 to those present in ATO, was created and tested in Drosophila (FIG. 8, Panel B). Seventeen out of 24 independent transgenic lines showed no neural induction. The other seven showed very weak induction with the dppGal4 driver. Mis-expression of the strongest NGN^{H2ATO} transgenic line induces a maximum of two bristles along the A-P axis of the wing per fly in 50% of the flies. Quantitative analysis shows that, unlike ATO, NGN^{H2ATO} induces an average of 0.8 bristles along A-P axis per fly (n=30, FIG. 8, Panel C). These data indicate that the group-specific motif in Helix2 of ATO does not encode proneural activity in Drosophila.

Conversely, we generated a chimeric protein, named ATO^{H2NGN}, exchanging the five group-specific amino acids in Helix2 of ATO to those found in NGN1 (FIG. 8, Panel B). Injection of 100 pg of ATO^{H2NGN} mRNA causes ectopic N-tubulin expression (FIG. 8, Panel D), indistinguishable from the injection of NGN1. These data suggest that the ATO^{H2NGN} mutant recovers the activity of NGN1 in Xenopus. Moreover, just like the injection of NGN1 and ATO^{bNGN} co-injection of 100 pg of ATO^{H2NGN} and 250 pg of X-MyT1 mRNAs results in synergy and very strong ectopic N-tubulin expression (FIG. 8, Panel E), suggesting that ATO^{H2NGN} and ATO^{bNGN} use the same mechanism of action as NGN1. Taken together, the mutational analysis results agree with the predictions of the ET analysis indicating that the group-specific residues in the Helix2 are sufficient for neuronal induction in Xenopus but not in Drosophila.

To visualize the location of the two motifs of the basic and Helix2 domains in the three-dimensional (3-D) structure of bHLH proteins, we superimposed the positions of the residues we exchanged onto the 3-D structure of the MyoD protein (Davis et al., 1989). The side chains of the residues in the basic domain form a continuous face pointing away from DNA and available for protein interaction (FIG. 8, Panel F). Similarly, the residues in Helix 2 form a continuous face protruding away from the dimerization partner (FIG. 8, Panel G). The computational modeling data indicate the strong possibility that the yet to be identified proteins bind to, or modify, both the basic and Helix2 domains, thereby regulating the specific activities of bHLH proteins.

### Example 4

### Loss of Hath1 Expression Induces an Aggressive Behavior in Merkel Carcinoma Cells

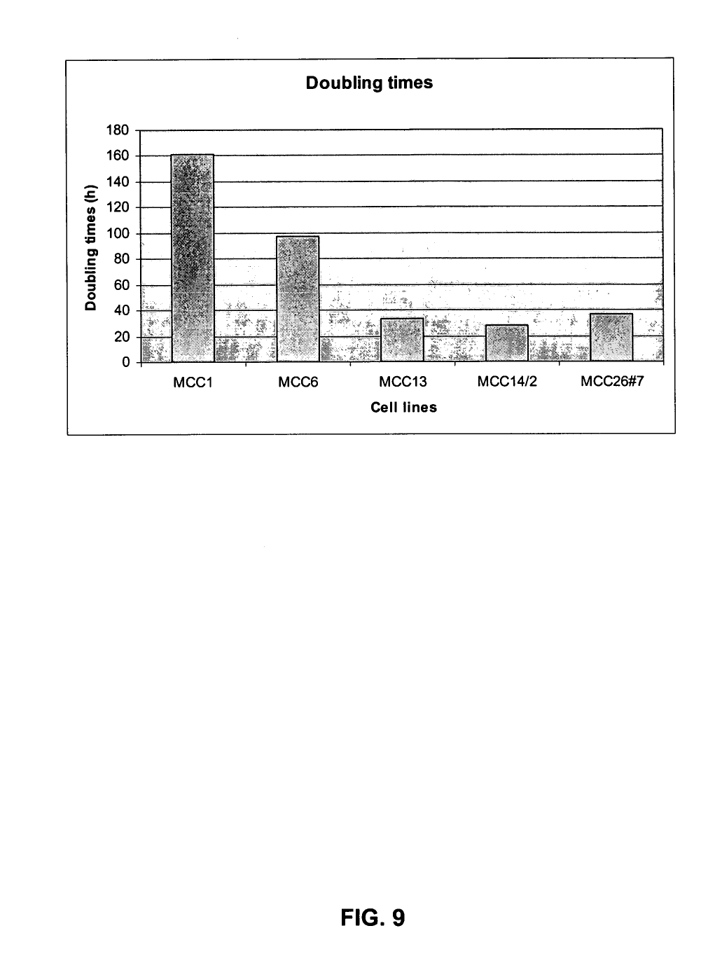

To determine if the expression of the human atonal homologue Hath1 can be correlated with the aggressive behavior of Merkel Cell Carcinoma (MCC) cells, we examined the replication rates of MCC cell lines which lack Hath1 expression (MCC13, MCC14 and MCC26) with those which still express Hath1 (MCC1 and MCC6). We found that MCC lines still expressing Hath1 have doubling times between 100 and 160 hours (FIG. 9).

In contrast, MCC lines which lack Hath1 are much more aggressive and have double times ranging between 28 and 36 hours. These data support the notion that loss of Hath1 expression contributes to the aggressive behavior of MCC cells.

### REFERENCES

Artavanis-Tsakonas S., M. D. Rand and R. J. Lake (1999) Notch signaling: cell fate control and signal integration in development. Science 284, 770-776.
Bailey A. M. and J. W. Posakony (1995) Suppressor of hairless directly activates transcription of enhancer of split complex genes in response to Notch receptor activity. Genes Dev. 9, 2609-2622.
Begbie J., M. Ballivet and A. Graham (2002) Early steps in the production of sensory neurons by the neurogenic placodes. Mol. Cell Neurosci. 21, 502-511.
Bellefroid E. J., C. Bourguignon, T. Hollemann, Q. Ma, D. J. Anderson, C. Kintner and T. Pieler (1996) X-MyT1, a Xenopus C2HC-type zinc finger protein with a regulatory function in neuronal differentiation. Cell 87, 1191-1202.
Ben-Arie N., B. A. Hassan, N. A. Bermingham, D. M. Malicki, D. Armstrong, M. Matzuk, H. J. Bellen and H. Y. Zoghbi (2000) Functional conservation of atonal and Math1 in the CNS and PNS. Development 127, 1039-1048.
Bertrand N., D. S. Castro and F. Guillemot (2002) Proneural genes and the specification of neural cell types. Nat. Rev. Neurosci. 3, 517-530.
Brown N. L., S. Kanekar, M. L. Vetter, P. K. Tucker, D. L. Gemza and T. Glaser (1998) Math5 encodes a murine basic helix-loop-helix transcription factor expressed during early stages of retinal neurogenesis. Development 125, 4821-4833.
Bush A., Y. Hiromi and M. Cole (1996) Biparous: a novel bHLH gene expressed in neuronal and glial precursors in Drosophila. Dev. Biol. 180, 759-772.
Chitnis A., D. Henrique, J. Lewis, D. Ish-Horowicz and C. Kintner (1995) Primary neurogenesis in Xenopus embryos regulated by a homologue of the Drosophila neurogenic gene Delta. Nature 375, 761-766.
Coffman C., W. Harris and C. Kintner (1990) Xotch, the Xenopus homolog of Drosophila notch. Science 249, 438-441.
Davis R. L., P. F. Cheng, A. B. Lassar, M. Thayer, S. Tapscott and H. Weintraub (1989) MyoD and achaete-scute: 4-5 amino acids distinguishes myogenesis from neurogenesis. Princess Takamatsu Symp. 20, 267-278.
Devereux J., P. Haeberli and 0. Smithies (1984) A comprehensive set of sequence analysis programs for the VAX. Nucleic Acids Res. 12, 387-395.
Feng D. F. and R. F. Doolittle (1987) Progressive sequence alignment as a prerequisite to correct phylogenetic trees. J. Mol. Evol. 25, 351-360.
Fode C., G. Gradwohl, X. Morin, A. Dierich, M. LeMeur, C. Goridis, P. Gautier, V. Ledent, M. Massaer, C. Dambly-Chaudiere and A. Ghysen (1997) tap, a Drosophila bHLH gene expressed in chemosensory organs. Gene 191, 15-21.
Goulding S. E., P. zur Lage and A. P. Jarman (2000) amos, a proneural gene for Drosophila olfactory sense organs that is regulated by lozenge. Neuron 25, 69-78.
Harland R. M. (1991) In situ hybridization: an improved whole-mount method for Xenopus embryos. Methods Cell Biol. 36, 685-695.
Hassan B. A. and H. J. Bellen (2000) Doing the MATH: is the mouse a good model for fly development? Genes Dev. 14, 1852-1865.
Helms A. W., K. Gowan, A. Abney, T. Savage and J. E. Johnson (2001) Overexpression of MATH1 disrupts the coordination of neural differentiation in cerebellum development. Mol. Cell. Neurosci. 17, 671-682.
Huang M. L., C. H. Hsu and C. T. Chien (2000) The proneural gene amos promotes multiple dendritic neuron formation in the Drosophila peripheral nervous system. Neuron 25, 57-67.
Jarman A. P., Y. Grau, L. Y. Jan and Y. N. Jan (1993) atonal is a proneural gene that directs chordotonal organ formation in the Drosophila peripheral nervous system. Cell 73, 1307-1321.
Jennings B., A. Preiss, C. Delidakis and S. Bray (1994) The Notch signaling pathway is required for Enhancer of split bHLH protein expression during neurogenesis in the Drosophila embryo. Development 120, 3537-3548.
Kanekar S., M. Perron, R. Dorsky, W. A. Harris, L. Y. Jan, Y. N. Jan and M. L. Vetter (1997) Xath5 participates in a network of bHLH genes in the developing Xenopus retina. Neuron 19, 981-994.
Kim P., A. W. Helms, J. E. Johnson and K. Zimmerman (1997) XATH-1, a vertebrate homolog of Drosophila atonal, induces a neuronal differentiation within ectodermal progenitors. Dev. Biol. 187, 1-12.
Kintner C. R. and D. A. Melton (1987) Expression of Xenopus N-CAM RNA in ectoderm is an early response to neural induction. Development 99, 311-325.
Lecourtois M. and F. Schweisguth (1995) The neurogenic suppressor of hairless DNA-binding protein mediates the transcriptional activation of the enhancer of split complex genes triggered by Notch signaling. Genes Dev. 9, 2598-2608.
Lichtarge O., H. R. Bourne and F. E. Cohen (1996a) Evolutionarily conserved Galphabetagamma binding surfaces support a model of the G protein-receptor complex. Proc. Natl. Acad. Sci. U.S.A. 93, 7507-7511.
Lichtarge O., H. R. Bourne and F. E. Cohen (1996b) An evolutionary trace method defines binding surfaces common to protein families. J. Mol. Biol. 257, 342-358.
Ma Q., C. Fode, F. Guillemot and D. J. Anderson (1999) Neurogenin1 and neurogenin2 control two distinct waves of neurogenesis in developing dorsal root ganglia. Genes Dev. 13, 1717-1728.
Ma Q., C. Kintner and D. J. Anderson (1996) Identification of neurogenin, a vertebrate neuronal determination gene. Cell 87, 43-52.
Madabushi S., H. Yao, M. Marsh, D. M. Kristensen, A. Philippi, M. E. Sowa and O. Lichtarge (2002) Structural clusters of evolutionary trace residues are statistically significant and common in proteins. J. Mol. Biol. 316, 139-154.
Mardon G., N. M. Solomon and G. M. Rubin (1994) dachshund encodes a nuclear protein required for normal eye and leg development in Drosophila. Development 120, 3473-3486.
Mayor R., R. Morgan and M. G. Sargent (1995) Induction of the prospective neural crest of Xenopus. Development 121, 767-777.
Nieuwkoop P. D. and J. Faber (1994) Normal table of Xenopus laevis. In (New York, Garland Publishing).
Onrust R., P. Herzmark, P. Chi, P. D. Garcia, O. Lichtarge, C. Kingsley and H. R. Bourne (1997) Receptor and betagamma binding sites in the alpha subunit of the retinal G protein transducin. Science 275, 381-384.
Oschwald R., K. Richter and H. Grunz (1991) Localization of a nervous system-specific class II beta-tubulin gene in Xenopus laevis embryos by whole-mount in situ hybridization. Int. J. Dev. Biol. 35, 399-405.
Rupp R. A., L. Snider and H. Weintraub (1994) Xenopus embryos regulate the nuclear localization of XMyoD. Genes Dev. 8, 1311-1323.
Sowa M. E., W. He, K. C. Slep, M. A. Kercher, O. Lichtarge and T. G. Wensel (2001) Prediction and confirmation of a site critical for effector regulation of RGS domain activity. Nat. Struct. Biol. 8, 234-237.
Sowa M. E., W. He, T. G. Wensel and O. Lichtarge (2000) A regulator of G protein signaling interaction surface linked to effector specificity. Proc. Natl. Acad. Sci. U.S.A. 97, 1483-1488.
Turner D. L. and H. Weintraub (1994) Expression of archaete-scute homolog 3 in Xenopus embryos converts ectodermal cells to neural fate. Genes Dev. 8, 1434-1447.
Vleminckx K., E. Wong, K. Guger, B. Rubinfeld, P. Polakis and B. M. Gumbiner (1997) Adenomatous polyposis coli tumor suppressor protein has signaling activity in Xenopus laevis embryos resulting in the induction of an ectopic dorsoanterior axis. J. Cell Biol. 136, 411-420.
Wang V. Y., B. A. Hassan, H. J. Bellen and H. Y. Zoghbi (2002) Drosophila atonal fully rescues the phenotype of Math1 null mice: new functions evolve in new cellular contexts. Curr. Biol. 12, 1611-1616.

1. A biological active artificial polypeptide comprising a domain selected from the group consisting of SEQ ID NO:1, SEQ ID NO:2, SEQ ID NO:3, SEQ ID NO:4, SEQ ID NO:5, SEQ ID NO:6, and SEQ ID NO:7.

2. A method of modulating neural precursor cell selection comprising the step of: administering the artificial polypeptide according to claim 1.

3. A method of modulating neural precursor cell selection comprising the step of: binding an antibody to a polypeptide of claim 1.

4. A method to specify the neuronal lineage identity of stem cells comprising the step of:
selecting a polypeptide of claim 1 to promote development of a specific stem cell.

5. A method of selecting inhibitors against a domain of a peptide comprising the steps of:
measuring the biological activity of a polypeptide according to claim 1 and
selecting the peptides that block the biological activity.

6. A method to induce MyT1 expression in a cell comprising the steps of:
admixing a polypeptide according to claim 1, wherein the polypeptide comprises a domain selected from the group consisting of SEQ ID NO: 1 and SEQ ID NO:3, and a cell, and
inducing MyT1 expression.

7. A method to induce expression of a member of the SENS family in a cell comprising the steps of:
admixing a polypeptide according to claim 1, wherein the polypeptide comprises a domain selected from the group consisting of SEQ ID NO:2 and SEQ ID NO:4, and a cell, and
inducing expression of a member of the SENS family.

8. The method of claim 7, wherein the member of the SENS family is Gfi-1.

9. A method to induce expression of a sensory organ precursor in a cell comprising the steps of:
admixing a polypeptide according to claim 1, wherein the polypeptide comprises a domain selected from the group consisting of SEQ ID NO:1, SEQ ID NO:3, SEQ ID NO:5, SEQ ID NO:6 and SEQ ID NO:7, and a cell, and
inducing expression of a sensory organ precursor.

10. A method to induce expression of a vertebrate inner hair cell comprising the steps of:
admixing a polypeptide according to claim 1, wherein the polypeptide comprises a domain selected from the group consisting of SEQ ID NO:2 and SEQ ID NO:4, and a cell, and
inducing vertebrate inner hair cell.

11. The method of claim 10, wherein said vertebrate is a mammal.

12. A method of treating cancer in a patient comprising the step of:
administering a therapeutically effective amount of a polypeptide according to claim 1, wherein the polypeptide comprises a domain selected from the group consisting of SEQ ID NO:1, SEQ ID NO:3, SEQ ID NO:5, SEQ ID NO:6 and SEQ ID NO:7 or an antibody against said domain to a patient in need thereof.

13. The method of claim 12, wherein the polypeptide comprises a domain selected from the group consisting of SEQ ID NO:2 and SEQ ID NO:4 or an antibody against said domain.

14. A method of treating an animal with a deficiency in cerebellar granule neurons or their precursors comprising delivery of a therapeutically effective amount of a polypeptide comprising a domain selected from the group consisting of SEQ ID NO:2 and SEQ ID NO:4 to a cell of said animal.

15. A method promoting mechanoreceptive cell growth in an animal comprising delivery of a therapeutically effective amount of a polypeptide comprising a domain selected from the group consisting of SEQ ID NO:2 and SEQ ID NO:4 to a cell of said animal.

16. A method of generating inner ear hair cells in an animal comprising delivery of a therapeutically effective amount of a polypeptide comprising a domain selected from the group consisting of SEQ ID NO:2 and SEQ ID NO:4 to a cell of said animal.

17. A method of treating an animal for hearing impairment comprising delivery of a therapeutically effective amount of a polypeptide comprising a domain selected from the group consisting of SEQ ID NO:2 and SEQ ID NO:4 to a cell of said animal.

18. A method according to claim 14, wherein said animal is a mammal.

19. A method according to claim 14, wherein said delivery is realized by in situ synthesis of said polypeptide.

20. A method according to claim 15, wherein said animal is a mammal.

21. A method according to claim 15, wherein said delivery is realized by in situ synthesis of said polypeptide.

22. A method according to claim 16, wherein said animal is a mammal.

23. A method according to claim 16, wherein said delivery is realized by in situ synthesis of said polypeptide.

24. A method according to claim 17, wherein said animal is a mammal.

25. A method according to claim 17, whereby said delivery is realized by in situ synthesis of said polypeptide.
